(19) United States
(12) Patent Application Publication
(10) Pub. No.: US 2019/0056383 A1
(43) Pub. Date: Feb. 21, 2019

(54) BIOLOGICAL SAMPLE EVALUATION SYSTEM, BIOLOGICAL SAMPLE EVALUATION METHOD, AND BIOLOGICAL SAMPLE EVALUATION CONTROL PROGRAM

(71) Applicant: FUJIFILM Corporation, Tokyo (JP)

(72) Inventor: Naoko YAMAGUCHI, Ashigarakami-gun (JP)

(73) Assignee: FUJIFILM Corporation, Tokyo (JP)

(21) Appl. No.: 16/169,059

(22) Filed: Oct. 24, 2018

Related U.S. Application Data
(63) Continuation of application No. PCT/JP2017/014165, filed on Apr. 5, 2017.

(30) Foreign Application Priority Data
Apr. 28, 2016 (JP) 2016-090458

(51) Int. Cl. G01N 33/50; A01N 1/02

(52) U.S. Cl. CPC G01N 33/5008; A01N 1/0284; A01N 1/0205

(57) ABSTRACT

Processes are sequentially performed on a plurality of biological samples of an identical type from a reference time, and the plurality of biological samples are sequentially evaluated from a time point at which a preset time elapses from the reference time, in which a time interval of each process on each of the biological samples is set to a time interval which is equal to or more than a shortest process operation time of the processing apparatus, and a time interval of each evaluation of each of the biological samples is set to a time interval which is equal to or more than a shortest evaluation operation time of the evaluation apparatus and is obtained by adding or subtracting a desired time interval shorter than the shortest evaluation operation time to or from the time interval of each process.

### CROSS-REFERENCE TO RELATED APPLICATIONS

This application is a Continuation of PCT International Application No. PCT/JP2017/014165 filed on Apr. 5, 2017, which claims priority under 35 U.S.C § 119(a) to Japanese Patent Application No. 2016-090458 filed on Apr. 28, 2016. Each of the above application(s) is hereby expressly incorporated by reference, in its entirety, into the present application.

### BACKGROUND OF THE INVENTION

### 1. Field of the Invention

The present invention relates to a biological sample evaluation system, a biological sample evaluation method, and a non-transitory computer readable recording medium storing a biological sample evaluation control program capable of evaluating a biological sample having undergone a predetermined process at a desired time interval.

### 2. Description of the Related Art

In the related art, in biology, a test is frequently performed in which a predetermined process is performed on biological tissue, and a nature change after the process is evaluated. Examples of the process include addition of a substance and irradiation with light, and an example of evaluation includes observation of morphology using a microscope.

Specifically, there is a case where a process such as addition of a chemical is performed on a biological sample, a temporal change of the biological sample is observed by performing evaluation (for example, capturing of an image of the biological sample) at a desired time interval from the time at which a predetermined time elapses, and thus an effect of the chemical is checked.

### SUMMARY OF THE INVENTION

Here, in a case where a temporal change of a biological sample is observed as described above, in the related art, a process may be performed on a single biological sample, and evaluation may be performed at a desired time interval from the time at which a predetermined time elapses.

However, in a case where a desired time interval for evaluation is shorter than the shortest evaluation operation time required for evaluation in an evaluation apparatus, realization thereof is not possible. For example, in a case where a morphological change after a chemical is added to a biological sample is observed with a microscope of the evaluation apparatus, processes such as movement of the biological sample between apparatuses, focusing of the microscope, and adjustment of an exposure amount of the microscope are necessary between certain observation and the next observation, and a predetermined time (for example, 60 seconds) (shortest evaluation operation time) are required therebetween.

On the other hand, in a case where a chemical is toxic, a reaction after addition of the chemical may occur abruptly, and thus it is necessary to perform observation at very short time intervals in order to elucidate the mechanism. In this case, a desired time interval for observation may be longer than the shortest evaluation operation time of an evaluation apparatus, and cannot be realized in a process and an evaluation for a single biological sample. JP1993-080057A (JP-H5-080057A) and JP2014-206381A propose a system which automatically controls procedures from a process for a biological sample to evaluation, but does not propose any control method in a case where the shortest evaluation operation time of an evaluation apparatus is longer than a desired time interval as described above.

In light of the problem, an object of the present invention is to provide a biological sample evaluation system, a biological sample evaluation method, and a non-transitory computer readable recording medium storing a biological sample evaluation control program capable of obtaining an evaluation result at a desired time interval even in a case where the shortest evaluation operation time of an evaluation apparatus is longer than the desired time interval for observation.

According to an aspect of the present invention, there is provided a biological sample evaluation system comprising a processing apparatus that performs an identical process on a plurality of biological samples of an identical type; an evaluation apparatus that evaluates the plurality of biological samples; and a control apparatus that controls a time interval of each process on each of the biological samples in the processing apparatus and a time interval of each evaluation of each biological sample in the evaluation apparatus, in which the control apparatus sets the time interval of each process to a time interval which is equal to or more than a shortest process operation time of the processing apparatus, sets the time interval of each evaluation to a time interval which is equal to or more than a shortest evaluation operation time of the evaluation apparatus and is obtained by adding or subtracting a desired time interval shorter than the shortest evaluation operation time to or from the time interval of each process, sequentially performs processes on the respective biological samples at the time interval of each process from a reference time, and sequentially evaluates the respective biological samples at the time interval of each evaluation from a time point at which a preset time elapses from the reference time.

Here, the “preset elapse time” includes zero seconds.

In the biological sample evaluation system according to the aspect of the present invention, the control apparatus preferably acquires the shortest process operation time and the shortest evaluation operation time, and sets the time interval of each process and the time interval of each evaluation on the basis of the acquired shortest process operation time and shortest evaluation operation time.

In the biological sample evaluation system according to the aspect of the present invention, the control apparatus preferably has an operation mode in which the time interval of each process and the time interval of each evaluation are set such that a time required to complete all processes and evaluations for the plurality of biological samples is shortest.

In the biological sample evaluation system according to the aspect of the present invention, the control apparatus has an operation mode in which the time interval of each process and the time interval of each evaluation are set such that a time required to complete all processes and evaluations for the plurality of biological samples is any time designated in advance.

In the biological sample evaluation system according to the aspect of the present invention, the control apparatus preferably has an operation mode in which the time interval of each process and the time interval of each evaluation are set such that a time required for the process and the evaluation for a single biological sample is equal to or less than a preset time.

In the biological sample evaluation system according to the aspect of the present invention, the control apparatus preferably has an operation mode in which the time interval of each process and the time interval of each evaluation are set such that a total use time of the processing apparatus is equal to or less than a preset time.

In the biological sample evaluation system according to the aspect of the present invention, the control apparatus preferably has an operation mode in which the time interval of each process and the time interval of each evaluation are set such that a total use time of the evaluation apparatus is equal to or less than a preset time.

In the biological sample evaluation system according to the aspect of the present invention, in a case where the control apparatus is not able to determine the time interval of each process and the time interval of each evaluation satisfying a condition of the operation mode, the control apparatus preferably performs a preset timing error operation.

In the biological sample evaluation system according to the aspect of the present invention, the control apparatus preferably performs a warning operation as the timing error operation.

In the biological sample evaluation system according to the aspect of the present invention, as the timing error operation, the control apparatus preferably displays the time interval of each process and the time interval of each evaluation which are realizable on a display apparatus.

In the biological sample evaluation system according to the aspect of the present invention, in a case where the processing apparatus or the evaluation apparatus causes an error, the control apparatus preferably performs a preset apparatus error operation.

In the biological sample evaluation system according to the aspect of the present invention, the control apparatus preferably performs a yarning operation as the apparatus error operation.

In the biological sample evaluation system according to the aspect of the present invention, the control apparatus preferably stops the processing apparatus and the evaluation apparatus as the apparatus error operation.

In the biological sample evaluation system according to the aspect of the present invention, in a case where either the processing apparatus or the evaluation apparatus causes an error, the control apparatus preferably stops one apparatus, and continuously operates the other apparatus.

According to another aspect of the present invention, there is provided a biological sample evaluation method comprising causing a processing apparatus to sequentially perform processes on a plurality of biological samples of an identical type from a reference time; and causing an evaluation apparatus to sequentially evaluate the plurality of biological samples from a time point at which a preset time elapses from the reference time, in which a time interval of each process on each of the biological samples is set to a time interval which is equal to or more than a shortest process operation time of the processing apparatus, and a time interval of each evaluation of each of the biological samples is set to a time interval which is equal to or more than a shortest evaluation operation time of the evaluation apparatus and is obtained by adding or subtracting a desired time interval shorter than the shortest evaluation operation time to or from the time interval of each process.

According to still aspect of the present invention, there is provided a non-transitory computer readable recording medium storing a biological sample evaluation control program for realizing a function of a control apparatus in a biological sample evaluation system comprising a processing apparatus that performs an identical process on a plurality of biological samples of an identical type, an evaluation apparatus that evaluates the plurality of biological samples, and the control apparatus that controls a time interval of each process on each of the biological samples in the processing apparatus and a time interval of each evaluation of each biological sample in the evaluation apparatus, the program causing the control apparatus to execute a procedure of setting the time interval of each process to a time interval which is equal to or more than a shortest process operation time of the processing apparatus a procedure of setting the time interval of each evaluation to a time interval which is equal to or more than a shortest evaluation operation time of the evaluation apparatus and is obtained by adding or subtracting a desired time interval shorter than the shortest evaluation operation time to or from the time interval of each process; a procedure of sequentially performing processes on the respective biological samples at the time interval of each process from a reference time; and a procedure of sequentially evaluating the respective biological samples at the time interval of each evaluation from a time point at which a preset time elapses from the reference time.

According to the biological sample evaluation system, the biological sample evaluation method, and the non-transitory computer readable recording medium storing the biological sample evaluation control program according to the aspects of the present invention, processes are sequentially performed on a plurality of biological samples of an identical type from a reference time, and the plurality of biological samples are sequentially evaluated from a time point at which a preset time elapses from the reference time. In this case, a time interval of each process on each of the biological samples is set to a time interval which is equal to or more than a shortest process operation time of a processing apparatus, and a time interval of each evaluation of each of the biological samples is set to a time interval which is equal to or more than a shortest evaluation operation time of an evaluation apparatus and is obtained by adding or subtracting a desired time interval shorter than the shortest evaluation operation time to or from the time interval of each process. By setting a time interval of each process and a time interval of each evaluation as mentioned above, it is possible to obtain an evaluation result at a desired time interval even in a case where a shortest evaluation operation time of an evaluation apparatus is longer than the desired time interval for observation.

### BRIEF DESCRIPTION OF THE DRAWINGS

FIG. 1 is a block diagram illustrating a schematic configuration of a biological sample evaluation system according to an embodiment of the present invention.

FIG. 2 is a graph illustrating examples of a time interval of each process on each biological sample and a time interval of each evaluation of each biological sample.

FIG. 3 is a graph illustrating other examples of a time interval of each process on each biological sample and a time interval of each evaluation of each biological sample.

FIG. 4 is a flowchart for explaining a shortest-time operation mode.

FIG. 5 is a flowchart for explaining an any-time operation mode.

FIG. 6 is a flowchart for explaining an execution time restriction operation mode.

FIG. 7 is a flowchart for explaining an apparatus use time restriction operation mode.

### DESCRIPTION OF THE PREFERRED EMBODIMENTS

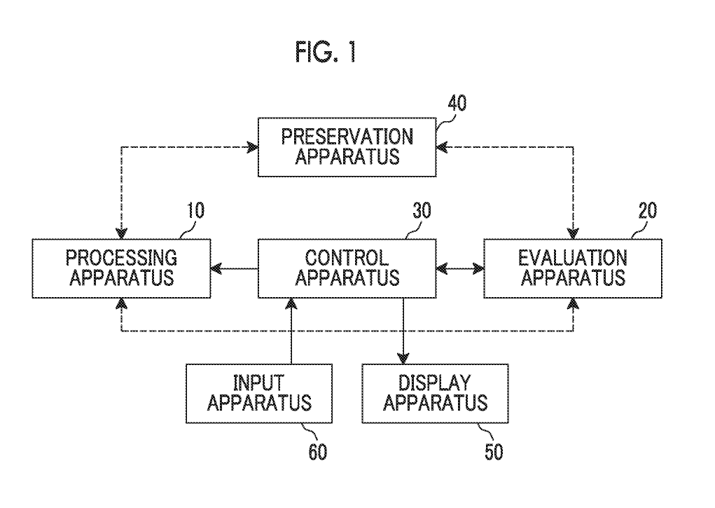

Hereinafter, with reference to the drawings, a detailed description will be made of a biological sample evaluation system according to an embodiment of the present invention. FIG. 1 is a block diagram illustrating a schematic configuration of a biological sample evaluation system of the present embodiment.

The biological sample evaluation system of the present embodiment comprises, as illustrated in FIG. 1, a processing apparatus 10, an evaluation apparatus 20, a control apparatus 30, a preservation apparatus 40, a display apparatus 50, and an input apparatus 60.

First, in a case where a biological sample is evaluated in the biological sample evaluation system of the present embodiment, a plurality of biological samples of an identical type are prepared. The plurality of biological samples of an identical type are a cell group regarded to be substantially identical in evaluation of the nature or the like of the cell group. For example, in a case where a biological sample is a cell, a plurality of biological samples are cultured from a single cell strain, and are created by dispensing the single cell strain to a plurality of containers of an identical type in an identical amount. In a case where even biological samples cultured from a different cell strain and are regarded to be substantially identical in evaluation of the nature or the like of a cell group, the biological samples may be used as a plurality of biological samples of an identical type.

Examples of biological samples include pluripotent stem cells such as induced pluripotent stem cells (iPS) and embryonic stem cells (ES), cells of a nerve, skin, cardiac muscle, or liver differentiated and induced from a stem cell, or cells of skin, a retina, cardiac muscle, a blood corpuscle, a nerve, or an organ extracted from a human body. As a container, for example, a petri dish may be used, a multi-well plate may be used, and different wells of a single multi-well plate may be used.

Creation of a plurality of biological samples may be manually performed by a user, and may be automatically performed by using, for example, an apparatus configured with a mechanism sucking cells, a robot arm, and the like.

The processing apparatus 10 performs an identical process on a plurality of biological samples of an identical type. A process performed by the processing apparatus 10 includes, for example, addition of a chemical of which a working mechanism is desired to be examined, or a physical stimulus such as irradiation with light. However, a process is not limited to such a process, and any process may be performed as long as the process is a necessary process for evaluating a biological sample. A process on each biological sample is performed by automatically adding a chemical to a container storing the biological sample by using an automatic dispensing device or the like.

The evaluation apparatus 20 evaluates the plurality of biological samples created as described above. Specifically, the evaluation apparatus 20 of the present embodiment captures an image of each biological sample in order to evaluate a morphological change of the biological sample, and comprises, for example, a phase difference microscope, a differential interference microscope, a bright-field microscope, or a fluorescence microscope. Such a microscope comprises an imaging element such as a complementary metal-oxide semiconductor (CMOS) sensor or a charge-coupled device (CCD) sensor.

Evaluation of a biological sample is not limited to capturing an image of the biological sample as in the present embodiment, and may be to measure the intensity of fluorescent light emitted from the biological sample, and may be to detect light emitted from the biological sample so as to perform spectral analysis of the light.

The preservation apparatus 40 comprises an incubator, and preserves a plurality of biological samples. The biological samples stored in the preservation apparatus 40 are extracted from the preservation apparatus 40, and are supplied to the processing apparatus 10 and the evaluation apparatus 20, in a case where a process and evaluation are performed. The biological samples are moved between the processing apparatus 10 and the preservation apparatus 40, between the evaluation apparatus 20 and the preservation apparatus 40, and between the processing apparatus 10 and the evaluation apparatus 20, as necessary. The biological samples may be automatically moved by using a robot arm, and may be automatically moved by using a conveyer belt or a tum table.

The display apparatus 50 comprises a display device such as a liquid crystal display. The display apparatus 50 displays a selection screen for receiving selection of each operation mode which will be described later and displays an input window for receiving various selling inputs from a user under the control of the control apparatus 30. An image of a biological sample captured by the evaluation apparatus 20 and other evaluation results may be displayed.

The input apparatus 60 comprises an input device such as a keyboard or a mouse, and receives various setting inputs from a user.

The control apparatus 30 is configured with a computer comprising a central processing unit (CPU), a memory, and the like, and controls the entire biological sample evaluation system. The control apparatus 30 is an apparatus in which a biological sample evaluation control program according to an embodiment of the present invention is installed in the computer, and a function of the control apparatus 30 is realized by the CPU executing the program. The biological sample evaluation control program is recorded on a recording medium such as a digital versatile disc (DVD) or a compact disc read only memory (CD-ROM) and is distributed, and is installed in a computer from the recording medium. The biological sample evaluation control program is stored in a storage device of a server computer connected to a network or a network storage in an accessible state from the outside, and is downloaded to and installed in a computer in response to a request.

The control apparatus 30 controls a time interval of each process on each biological sample in the processing apparatus 10 and a time interval of each evaluation of each biological sample in the evaluation apparatus 20. Specifically, even in a case where the shortest evaluation operation time of the evaluation apparatus 20 is longer than a desired time interval for observation, the control apparatus 30 of the present embodiment controls a time interval of each process on each biological sample and a time interval of each evaluation of each biological sample by using a plurality of biological samples of an identical type such that the biological sample can be evaluated at the desired time interval.

Hereinafter, a description will be made of control of the control apparatus 30 in a case where a biological sample is evaluated every x seconds from the time at which T seconds elapses after a process on the biological sample is performed. In addition, x seconds is the desired time interval. Herein, it is assumed that n biological samples are prepared.

First, the control apparatus 30 sets a time interval t1 of each process on each biological sample in the processing apparatus 10 and a time interval t2 of each evaluation of each biological sample in the evaluation apparatus 20. The time interval t1 of each process is a time interval which is equal to or more than the shortest process operation time of the processing apparatus 10, and is a time interval which is set and input by a user. The shortest process operation time is the shortest time required for the processing apparatus 10 to perform a process on a single biological sample, The time interval t2 of each evaluation is a time interval which is equal to or more than the shortest evaluation operation time and is obtained by adding the desired time interval x to the time interval t1 of each process. In other words, t2=t1+x. The time interval t2 of each evaluation is also a time interval set and input by the user.

The control apparatus 30 sets the time interval t1 of each process and the time interval t2 of each evaluation on the basis of setting inputs from the user, controls the processing apparatus 10 to sequentially perform n processes on the respective biological samples at the time interval t1 from the reference time of 0 seconds, and also controls the evaluation apparatus 20 to sequentially perform n evaluations of the respective biological samples at the time interval t2 from the time at which T seconds elapses from the reference time of 0 seconds. The elapse time T may be zero seconds.

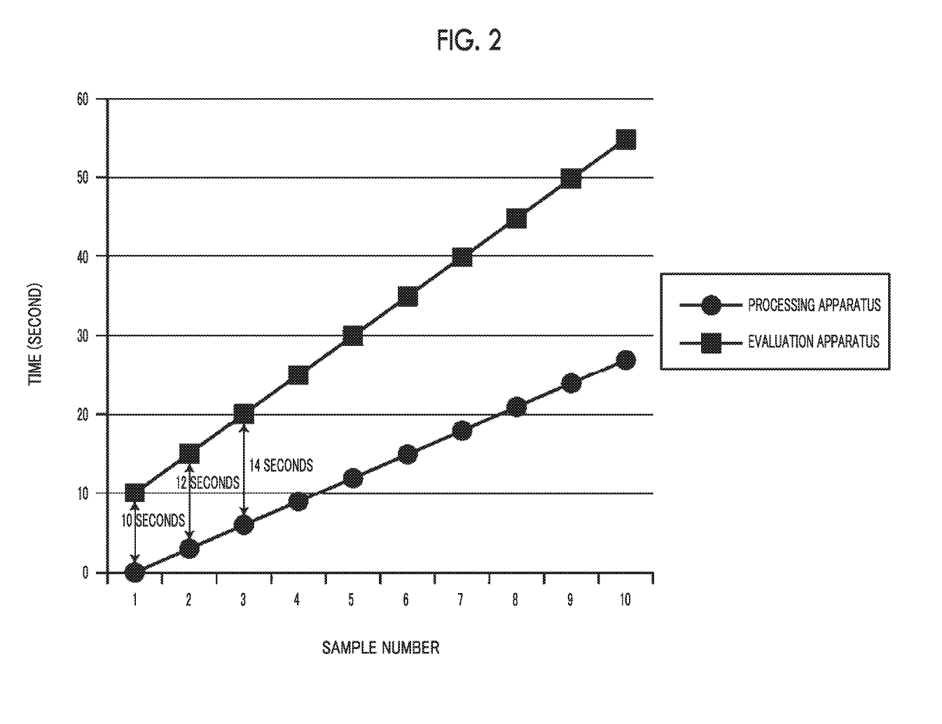

FIG. 2 is a graph illustrating process timings (indicated by black circles) and evaluation timings (indicated by black squares) for respective ten biological samples in a case where the ten biological samples are prepared, the time interval t1 of each process on each biological sample is set to 3 seconds, the time interval t2 of each evaluation of each biological sample is set to 5 seconds, and the elapse time T from the reference time is set to 10 seconds. Herein, the time interval t1 of each process is referred to as the shortest process operation time, and the time interval t2 of each evaluation is referred to as the shortest evaluation operation time.

According to each process timing and each evaluation timing illustrated in FIG. 2, for example, with respect to the biological sample with the sample number 1, an evaluation is performed at the time at which 10 seconds elapses after a process is performed; with respect to the biological sample with the sample number 2, an evaluation is performed at the time at which 12 seconds elapses after a process is performed; and, with respect to the biological sample with the sample number 3, an evaluation is performed at the time at which 14 seconds elapses after a process is performed. Therefore, an evaluation is substantially performed at a time interval of 2 seconds, that is, the evaluation can be performed at a time interval shorter than the shortest evaluation operation time.

In the above description, the time interval t2 of each evaluation is an interval obtained by adding the desired time interval x to the time interval t1 of each process, but is not limited thereto, and may be an interval obtained by subtracting the desired time interval x from the time interval t1 of each process. In other words, t2=t1−x. However, as described above, the time interval t2 is a time which is equal to or more than the shortest evaluation operation time.

In this case, the control apparatus 30 controls the processing apparatus 10 to sequentially perform n processes on the respective biological samples at the time interval t1 from the reference time of 0 seconds, and also controls the evaluation apparatus 20 to sequentially perform n evaluations of the respective biological samples at the time interval t2 from the time point of {T+(n−1)x} seconds.

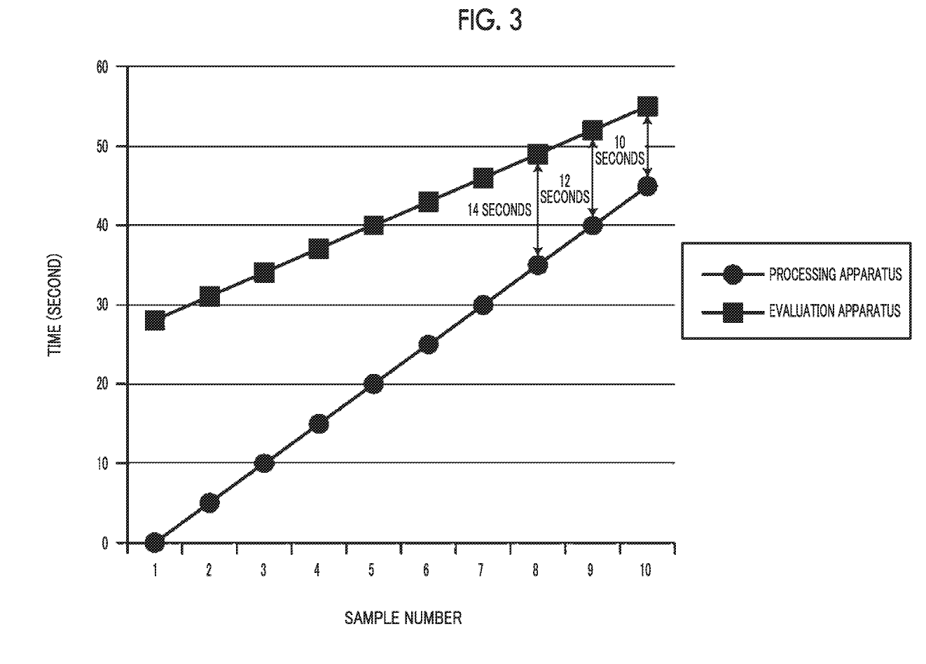

FIG. 3 is a graph illustrating process timings (indicated by black circles) and evaluation timings (indicated by black squares) for respective ten biological samples in a case where the ten biological samples are prepared, the time interval t1 of each process on each biological sample is set to 5 seconds, the time interval t2 of each evaluation of each biological sample is set to 3 seconds, and the elapse time T from the reference time is set to 28 seconds. Herein, the time interval t1 of each process is referred to as the shortest process operation time, and the time interval t2 of each evaluation is referred to as the shortest evaluation operation time.

According to each process tuning and each evaluation timing illustrated in FIG. 3, for example, with respect to the biological sample with the sample number 10, an evaluation is performed at the time at which 10 seconds elapses after a process is performed; with respect to the biological sample with the sample number 9, an evaluation is performed at the time at which 12 seconds elapses after a process is performed; and, with respect to the biological sample with the sample number 8, an evaluation is performed at the time at which 14 seconds elapses after a process is performed. Therefore, an evaluation is substantially performed at an interval of 2 seconds, that is, the evaluation can be performed at an interval shorter than the shortest evaluation operation time.

Here, in the examples illustrated in FIGS. 2 and 3, the time interval t1 of each process is set as the shortest process operation time, and the time interval t2 of each evaluation is set as the shortest evaluation operation time, but this is only an example, and, as described above, an evaluation can be performed at the desired time interval x in a case where the time interval t1 of each process and the time interval t2 of each evaluation are respectively equal to or more than the shortest process operation time and the shortest evaluation operation time, and satisfy a relationship of t2=t1+x or t2=t1−x.

Here, in a case where an evaluation result is desired to be obtained as soon as possible, or a constraint time of a human monitoring an evaluation is shortened, the time interval t1 of each process and the time interval t2 of each evaluation are preferably set such that the time required to complete all processes and evaluations for the respective biological samples is shortest.

In a case where the stability of the processing apparatus 10, the evaluation apparatus 20, or the entire biological sample evaluation system is checked, the stability of an operation is preferably checked by operating the biological sample evaluation system for a long period of time to some extent. In other words, the time interval t1 of each process and the time interval t2 of each evaluation are preferably set such that the time required to complete all processes and evaluations for the respective biological samples is any time designated by the user.

Therefore, the control apparatus 30 of the present embodiment has a basic operation mode for controlling the processing apparatus 10 and the evaluation apparatus 20 on the basis of the time interval t1 of each process and the time interval t2 of each evaluation which are set and input by the user, and also has various operation modes corresponding to the user's request. An operation mode selection screen is displayed on, for example, the display apparatus 50 in order to select various operation modes including the basic operation mode. The user selects any operation mode on the selection screen by using the input apparatus 60, and thus the control apparatus 30 sets the time interval t1 of each process and the time interval t2 of each evaluation for each biological sample corresponding to the operation mode.

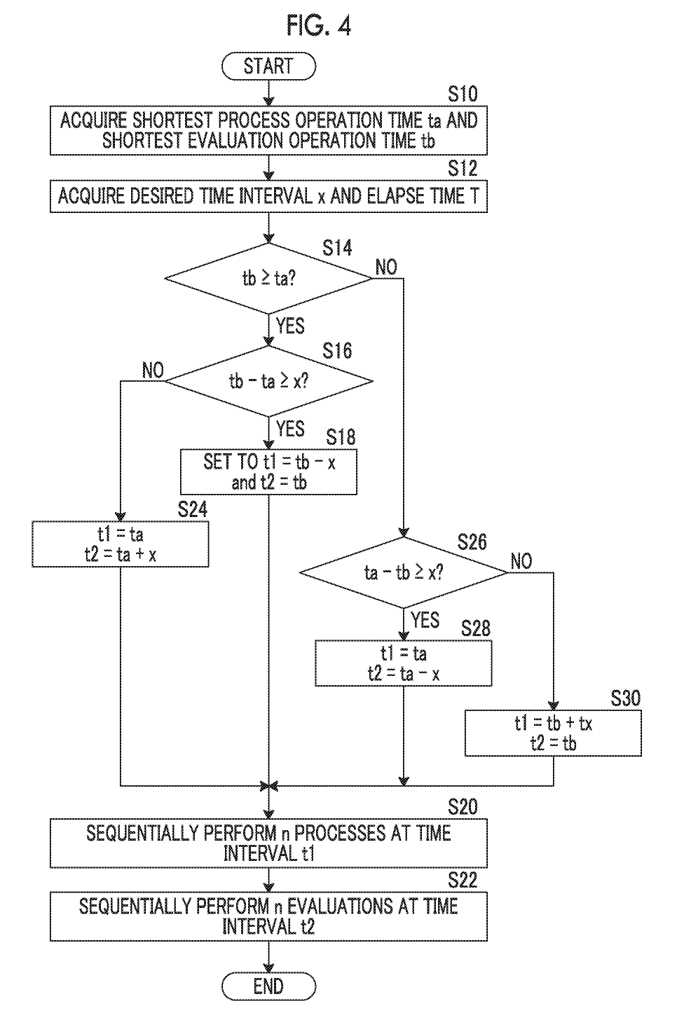

Hereinafter, a description will be made of a method of setting the time interval t1 of each process and the time interval t2 of each evaluation for each biological sample in each operation mode. First, with reference to a flowchart in FIG. 4, a description will be made of a shortest-time operation mode of setting the time interval t1 of each process and the time interval t2 of each evaluation such that the time required to complete all processes and evaluations for the respective biological samples is shortest.

In a case where the shortest-time operation mode is selected, the control apparatus 30 acquires a shortest process operation time ta and a shortest evaluation operation time tb (S10). The shortest process operation time ta and the shortest evaluation operation time tb may be set and input by the user by using the input apparatus 60, and may be stored in and acquired from a memory or the like of the processing apparatus 10, the evaluation apparatus 20, or the control apparatus 30. In a case where the user sets and inputs the times, an input window may be displayed on the display apparatus 50.

Next, the control apparatus 30 acquires the desired evaluation time interval x and the elapse time T from the reference time (S12). The desired evaluation time interval x and the elapse time T from the reference time may be set and input by the user by using the input apparatus 60, and may be stored in and acquired from the memory or the like of the control apparatus 30. In a case where the user sets and inputs the times, an input window may be displayed on the display apparatus 50.

The control apparatus 30 sets the time interval t1 of each process and the time interval t2 of each evaluation for each biological sample as follows on the basis of the shortest process operation time ta, the shortest evaluation operation time tb, the desired time interval x, and the elapse time T from the reference time.

In a case where tb≥ta and tb−ta≥x (YES in S14 and YES in S16), the control apparatus 30 sets the time interval t1 of each process on each biological sample to (tb−x), and sets the time interval t2 of each evaluation of each biological sample to tb (S18).

The control apparatus 30 controls the processing apparatus 10 to sequentially perform n processes on the respective biological samples at the time interval t1 (=(tb−x)) from the time point of 0 seconds (S20), and also controls the evaluation apparatus 20 to sequentially perform n evaluations of the respective biological samples at the time interval t2 (=tb) from the time at which T seconds elapses from the time point of 0 seconds (S22).

In a case where tb≥ta and tb−ta<x (YES in S14 and NO in S16), the control apparatus 30 sets the time interval t1 of each process on each biological sample to ta, and sets the time interval t2 of each evaluation of each biological sample to (ta+x) (S24).

The control apparatus 30 controls the processing apparatus 10 to sequentially perform n processes on the respective biological samples at the time interval t1 (=(ta)) from the time point of 0 seconds (S20), and also controls the evaluation apparatus 20 to sequentially perform n evaluations of the respective biological samples at the time interval t2 (=ta+x) from the time at which T seconds elapses from the time point of 0 seconds (S22).

In a case where tb<ta and ta−tb≥x (NO in S14 and YES in S26), the control apparatus 30 sets the time interval t1 of each process on each biological sample to ta, and sets the time interval t2 of each evaluation of each biological sample to (ta−x) (S28).

The control apparatus 30 controls the processing apparatus 10 to sequentially perform n processes on the respective biological samples at the time interval t1 (=(ta)) from the time point of 0 seconds (S20), and also controls the evaluation apparatus 20 to sequentially perform n evaluations of the respective biological samples at the time interval t2 (=ta−x) from the time at which {T+(n−1)x} seconds elapses from the time point of 0 seconds (S22).

In a case where tb<ta and ta−tb<x (NO in S14 and NO in S26), the control apparatus 30 sets the time interval t1 of each process on each biological sample to (tb+x), and sets the time interval t2 of each evaluation of each biological sample to tb (S30).

The control apparatus 30 controls the processing apparatus 10 to sequentially perform n processes on the respective biological samples at the time interval t1 (=tb+x) from the time point of 0 seconds (S20), and also controls the evaluation apparatus 20 to sequentially perform n evaluations of the respective biological samples at the time interval t2 (=tb) from the time at which {T+(n−1)x} seconds elapses from the time point of 0 seconds (S22)

In a case where the shortest-time operation mode is selected, the control apparatus 30 sets the time interval t1 of each process and the time interval t2 of each evaluation for each biological sample in the above-described way, and can thus make the time required to complete all processes and evaluations for the respective biological samples shortest at all times.

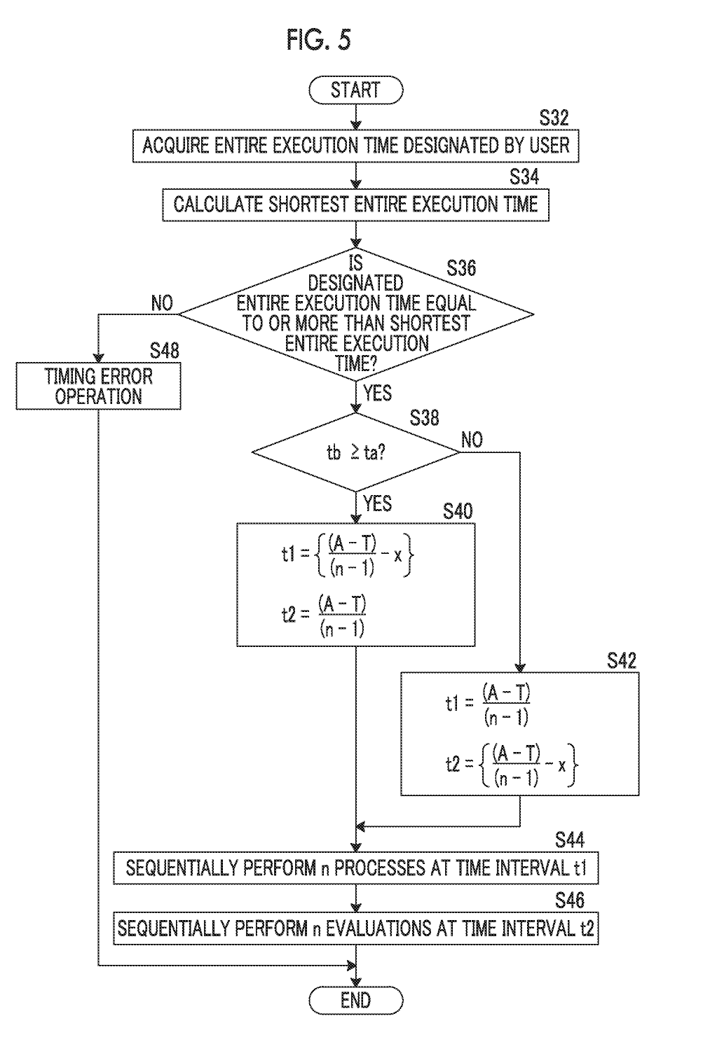

Next, with reference to a flowchart in FIG. 5, a description will be made of an any-time operation mode of setting the time interval t1 of each process and the time interval t2 of each evaluation such that the time required to complete all processes and evaluations for the respective biological samples is any time designated by the user.

In a case where the any-time operation mode is selected, the control apparatus 30 acquires any entire execution time A which is set and input by the user by using the input apparatus 60 (S32). The entire execution time A is the time required to complete all processes and evaluations for the respective biological samples. Also with respect to input of the entire execution time A, an input window may be displayed on the display apparatus 50.

Next, the control apparatus 30 calculates the shortest entire execution time of the biological sample evaluation system (S34). The shortest entire execution time is calculated by using the same method as that in the above-described shortest-time operation mode. In a case where tb≥ta and tb−ta≥x, the shortest entire execution time is {T+(n−1)tb} seconds, and, on the other hand, in a case where tb≥ta and tb−ta<x, the shortest entire execution time is {T+(n−1)(ta+x)} seconds. In a case where tb<ta and ta−tb≥x, the shortest entire execution time is {T+(n−1)ta} seconds, and, on the other hand, in a case where tb<ta and ta−tb<x, the shortest entire execution time is {T+(n−1)(tb+x)} seconds.

In a case where the entire execution time A designated by the user is equal to or more than the shortest entire execution time (YES in S36), the control apparatus 30 sets the time interval t1 of each process and the time interval t2 of each evaluation for each biological sample as follows.

In a case where tb≥ta (YES in S38), the control apparatus 30 sets the time interval t1 of each process on each biological sample to {(A−T)/(n−1)−x}, and sets the time interval t2 of each evaluation of each biological sample to (A−T)/(n−1) (S40).

The control apparatus 30 controls the processing apparatus 10 to sequentially perform n processes on the respective biological samples at the time interval t1 from the time point of 0 seconds (S44), and also controls the evaluation apparatus 20 to sequentially perform n evaluations of the respective biological samples at the time interval t2 from the time at which T seconds elapses from the time point of 0 seconds (S46).

In a case where tb<ta (NO in S38), the control apparatus 30 sets the time interval t1 of each process on each biological sample to (A−T)/(n−1), and sets the time interval t2 of each evaluation of each biological sample to {(A−T)/(n−1)−x}(S42).

The control apparatus 30 controls the processing apparatus 10 to sequentially perform n processes on the respective biological samples at the time interval t1 from the time point of 0 seconds (S44), and also controls the evaluation apparatus 20 to sequentially perform n evaluations of the respective biological samples at the time interval t2 from the time at which T seconds elapses from the time point of 0 seconds (S46).

On the other hand, in a case where the entire execution time A designated by the user is shorter than the shortest entire execution time, that is, the time interval t1 of each process and the time interval t2 of each evaluation satisfying the condition of the entire execution time A cannot be determined, the control apparatus 30 performs a timing error operation (S48).

As the timing error operation, the control apparatus 30 displays an error message or the like on the display apparatus 50 so as to perform a warning operation. The warning operation is not limited to display of an error message, and a warning sound may be issued, or an icon indicating an error may be displayed.

As the timing error operation, the time interval t1 of each process and the time interval t2 of each evaluation which are realizable may be displayed on the display apparatus 50. Specifically, for example, the time interval t1 of each process and the time interval t2 of each evaluation in the shortest-time operation mode may be displayed on the display apparatus 50, and a selection of whether or not the shortest-time operation mode is performed may be received.

According to the any-time operation mode, the biological sample evaluation system can be operated for any execution time designated by the user, and thus the stability of the operation of the system can be checked. In a case where the condition of the any-time operation mode cannot be satisfied, the user can perform appropriate adjustment by performing the timing error operation.

Next, a description will be made of an execution time restriction operation mode in which the time interval t1 of each process and the time interval t2 of each evaluation are set such that the time required for a process and an evaluation for a single biological sample is equal to or less than a preset time.

A biological sample such as a cell is preserved in the preservation apparatus 40 in which humidity, a temperature, and a carbon dioxide concentration are appropriately managed, but is temporarily extracted from the preservation apparatus 40 in a case where a process in the processing apparatus 10 and an evaluation in the evaluation apparatus 20 are performed. An environment outside the preservation apparatus 40 is often in the air and is different from an environment inside the preservation apparatus 40. In other words, a biological sample is exposed to an environment in which the biological sample hardly survives during a process in the processing apparatus 10 and an evaluation in the evaluation apparatus 20. In a case where a period of time in which a biological sample is exposed to the air is long, survival of the biological sample is influenced, and thus it is preferable to restrict the time required for a process and an evaluation for a single biological sample. The execution time restriction operation mode is an operation mode which is set in consideration of the above-described. circumstances.

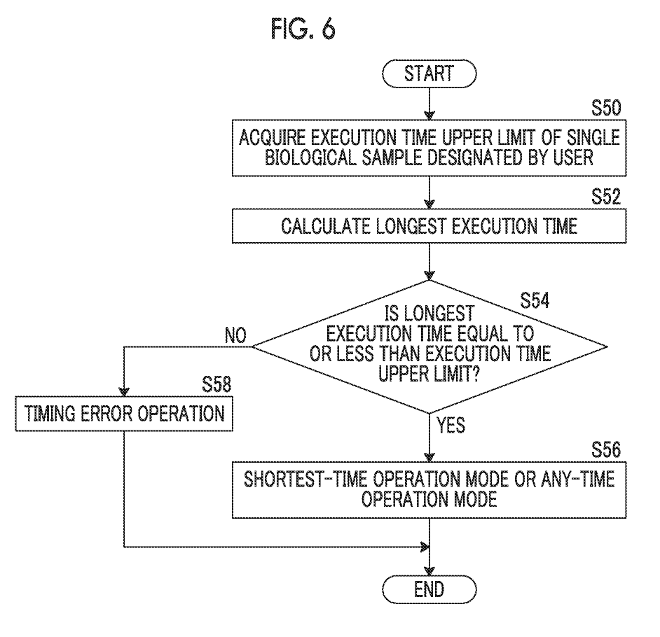

Hereinafter, the execution time restriction operation mode will be described with reference to a flowchart in FIG. 6.

In a case where the execution time restriction operation mode is selected, the control apparatus 30 acquires an execution time upper limit B which is set and input by the user by using the input apparatus 60 (S50).

Next, the control apparatus 30 calculates the longest execution time for a single biological sample (S52). The longest execution time is the longest execution time among execution times required for a process and an evaluation for each biological sample, and is {T+(n−1)x} seconds in a case where the elapse time T from the reference time is indicated by T, the number of biological samples is indicated by n, and the desired evaluation time interval is indicated by x.

In a case where the longest execution time is equal to or less than the execution time upper limit B designated by the user (YES in S54), the control apparatus 30 performs the shortest-time operation mode or the any-time operation mode. An operation mode to be performed of the shortest-time operation mode and the any-time operation mode may be set in advance, and may be selected on a selection screen by the user.

On the other hand, in a case where the longest execution time exceeds the execution time upper limit B designated by the user (NO in S54), that is, the time interval t1 of each process and the time interval t2 of each evaluation satisfying the condition of the execution time upper limit B cannot be determined, the control apparatus 30 performs a timing error operation (S58).

As the timing error operation, the control apparatus 30 displays an error message or the like on the display apparatus 50 so as to perform a warning operation. The warning operation is not limited to display of an error message, and a warning sound may be issued, or an icon indicating an error may be displayed.

As the timing error operation, a realizable execution time for a single biological sample may be displayed on the display apparatus 50. Specifically, for example, the above {T+(n−1)x} may be displayed on the display apparatus 50, and such a condition may be used without being changed, or a change of the elapse time T from the reference time, the number n of biological samples, or the desired evaluation time interval x may be received.

According to the execution time operation mode, it is possible to automatically determine whether or not the time required for a process and an evaluation for a single biological sample will be able to be equal to or less than a preset time, and, in a case where realization thereof is not possible, a user can perform appropriate adjustment.

Next, a description will be made of an apparatus use time restriction operation mode in which the time interval t1 of each process and the time interval t2 of each evaluation are set such that a total use time of the processing apparatus 10 or the evaluation apparatus 20 is equal to or less than a preset time.

There is a case where other users use the processing apparatus 10 or the evaluation apparatus 20 in other evaluations in addition to a single user. In this case, a process and an evaluation are preferably performed by restricting a total use time of the processing apparatus 10 or the evaluation apparatus 20. The apparatus use time restriction operation mode is an operation mode which is set in consideration of the above-described circumstances.

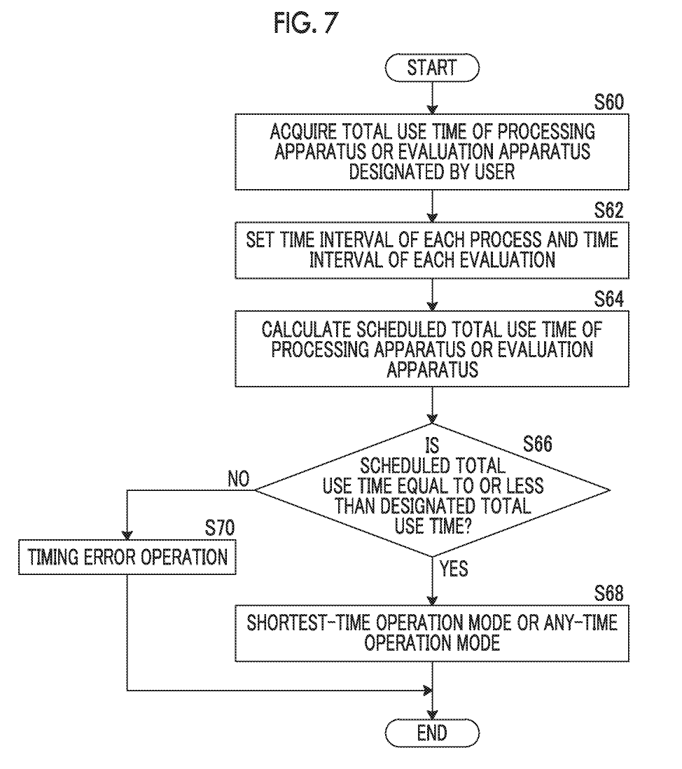

Hereinafter, the apparatus use time restriction operation mode will be described with reference to a flowchart in FIG. 7.

In a case where the apparatus use time restriction operation mode is selected, the control apparatus 30 acquires a total use time of the processing apparatus 10 or the evaluation apparatus 20 which is set and input by the user by using the input apparatus 60 (S60).

Next, the control apparatus 30 receives a selection of any one of the shortest-time operation mode and the any-time operation mode, and sets the time interval t1 of each process and the time interval t2 of each evaluation according to the selected operation mode (S62).

Next, the control apparatus 30 calculates a scheduled total use time of the processing apparatus 10 or the evaluation apparatus 20 on the basis of the set time interval t1 of each process or the set time interval t2 of each evaluation (S64). The scheduled total use time of the processing apparatus 10 is calculated as (n−1)t1, and the scheduled total use time of the evaluation apparatus 20 is calculated as (n−1)12.

In a case where the scheduled total use time of the processing apparatus 10 or the evaluation apparatus 20 is equal to or less than the total use time of the processing apparatus 10 or the evaluation apparatus 20 designated by the user (YES in S66), the control apparatus 30 performs the shortest-time operation mode or the any-time operation mode selected by the user.

On the other hand, in a case where the scheduled total use time of the processing apparatus 10 or the evaluation apparatus 20 exceeds the total use time of the processing apparatus 10 or the evaluation apparatus 20 designated by the user (NO in S66), that is, the time interval t1 of each process and the time interval t2 of each evaluation satisfying the condition of the total use time designated by the user cannot be determined, the control apparatus 30 performs a timing error operation (S70).

As the timing error operation, the control apparatus 30 displays an error message or the like on the display apparatus 50 so as to perform a warning operation. The warning operation is not limited to display of an error message, and a warning sound may be issued, or an icon indicating an error may be displayed.

As the timing error operation, a realizable total use time of the processing apparatus 10 or the evaluation apparatus 20 may be displayed on the display apparatus 50. Specifically, for example, the above (n−1)t1 or (n−1)t2 may be displayed on the display apparatus 50, and such a condition may be used without being changed, or a change of the number n of biological samples or the desired evaluation time interval x may be received.

According to the apparatus use time restriction operation mode, it is possible to automatically determine whether or not a total use time of the processing apparatus 10 or the evaluation apparatus 20 will be able to be equal to or less than a preset time, and, in a case where realization thereof is not possible, a user can perform appropriate adjustment. Consequently, it is possible to improve the usage efficiency of the processing apparatus 10 or the evaluation apparatus 20.

In the biological sample evaluation system of the embodiment, in a case where the processing apparatus 10 or the evaluation apparatus 20 wrongly operates or fails and thus causes an error, a preset apparatus error operation may be performed.

As the apparatus error operation, the control apparatus 30 displays an apparatus error message or the like on the display apparatus 50 so as to perform a warning operation. The warning operation is not limited to display of an apparatus error message, and a warning sound may be issued, or an icon indicating an apparatus error may be displayed.

Alternatively, as the apparatus error operation, the control apparatus 30 may stop the processing apparatus 10 or the evaluation apparatus 20. However, in a case where an apparatus error occurs in either the processing apparatus 10 or the evaluation apparatus 20, the one apparatus may be stopped, and the other apparatus may be continuously operated.

For example, in a case where an apparatus error occurs in only the evaluation apparatus 20, the evaluation apparatus 20 may be stopped, only a process may be continuously performed by the processing apparatus 10, and an evaluation may be performed by the user through visual observation. Alternatively, for example, in a case where an apparatus error occurs in only the processing apparatus 10, the processing apparatus 10 may be stopped, only an evaluation may be continuously performed by the evaluation apparatus 20, and only an evaluation of a biological sample having undergone an appropriate process may be referred to.

### EXPLANATION OF REFERENCES

10: processing apparatus

20: evaluation apparatus

30: control apparatus

40: preservation apparatus

50: display apparatus

60: input apparatus

What is claimed is:

1. A biological sample evaluation system comprising:
a processing apparatus that performs an identical process on a plurality of biological samples of an identical type;
an evaluation apparatus that evaluates the plurality of biological samples; and
a control apparatus that controls a time interval of each process on each of the biological samples in the processing apparatus and a time interval of each evaluation of each biological sample in the evaluation apparatus,
wherein the control apparatus sets the time interval of each process to a time interval which is equal to or more than a shortest process operation time of the processing apparatus, sets the time interval of each evaluation to a time interval which is equal to or more than a shortest evaluation operation time of the evaluation apparatus and is obtained by adding or subtracting a desired time interval shorter than the shortest evaluation operation time to or from the time interval of each process, sequentially performs processes on the respective biological samples at the time interval of each process from a reference time, and sequentially evaluates the respective biological samples at the time interval of each evaluation from a time point at which a preset time elapses from the reference time.

2. The biological sample evaluation system according to claim 1,
wherein the control apparatus acquires the shortest process operation time and the shortest evaluation operation time, and sets the time interval of each process and the time interval of each evaluation on the basis of the acquired shortest process operation time and shortest evaluation operation time.

3. The biological sample evaluation system according to claim 2,
wherein the control apparatus has an operation mode in which the time interval of each process and the time interval of each evaluation are set such that a time required to complete all processes and evaluations for the plurality of biological samples is shortest.

4. The biological sample evaluation system according to claim 2,
wherein the control apparatus has an operation mode in which the time interval of each process and the time interval of each evaluation are set such that a time required to complete all processes and evaluations for the plurality of biological samples is any time designated in advance.

5. The biological sample evaluation system according to claim 3,
wherein the control apparatus has an operation mode in which the time interval of each process and the time interval of each evaluation are set such that a time required to complete all processes and evaluations for the plurality of biological samples is any time designated in advance.

6. The biological sample evaluation system according to claim 1,
wherein the control apparatus has an operation mode in which the time interval of each process and the time interval of each evaluation are set such that a time required for the process and the evaluation for a single biological sample is equal to or less than a preset time.

7. The biological sample evaluation system according to claim 2,
wherein the control apparatus has an operation mode in which the time interval of each process and the time interval of each evaluation are set such that a time required for the process and the evaluation for a single biological sample is equal to or less than a preset time.

8. The biological sample evaluation system according to claim 3,
wherein the control apparatus has an operation mode in which the time interval of each process and the time interval of each evaluation are set such that a time required for the process and the evaluation for a single biological sample is equal to or less than a preset time.

9. The biological sample evaluation system according to claim 4,
wherein the control apparatus has an operation mode in which the time interval of each process and the time interval of each evaluation are set such that a time required for the process and the evaluation for a single biological sample is equal to or less than a preset time.

10. The biological sample evaluation system according to claim 1,
wherein the control apparatus has an operation mode in which the time interval of each process and the time interval of each evaluation are set such that a total use time of the processing apparatus is equal to or less than a preset time.

11. The biological sample evaluation system according to claim 1,
wherein the control apparatus has an operation mode in which the time interval of each process and the time interval of each evaluation are set such that a total use time of the evaluation apparatus is equal to or less than a preset time.

12. The biological sample evaluation system according to claim 4,
wherein, in a case where the control apparatus is not able to determine the time interval of each process and the time interval of each evaluation satisfying a condition of the operation mode, the control apparatus performs a preset timing error operation.

13. The biological sample evaluation system according to claim 12,
wherein the control apparatus performs a warning operation as the timing error operation.

14. The biological sample evaluation system according to claim 12,
wherein, as the timing error operation, the control apparatus displays the time interval of each process and the time interval of each evaluation which are realizable on a display apparatus.

15. The biological sample evaluation system according to claim 1,
wherein, in a case where the processing apparatus or the evaluation apparatus causes an error, the control apparatus performs a preset apparatus error operation.

16. The biological sample evaluation system according to claim 15,
wherein the control apparatus performs a warning operation as the apparatus error operation.

17. The biological sample evaluation system according to claim 15,
wherein the control apparatus stops the processing apparatus and the evaluation apparatus as the apparatus error operation.

18. The biological sample evaluation system according to claim 15,
wherein, in a case where either the processing apparatus or the evaluation apparatus causes an error, the control apparatus stops one apparatus, and continuously operates the other apparatus.

19. A biological sample evaluation method comprising:
causing a processing apparatus to sequentially perform processes on a plurality of biological samples of an identical type from a reference time; and
causing an evaluation apparatus to sequentially evaluate the plurality of biological samples from a time point at which a preset time elapses from the reference time,
wherein a time interval of each process on each of the biological samples is set to a time interval which is equal to or more than a shortest process operation time of the processing apparatus, and a time interval of each evaluation of each of the biological samples is set to a time interval which is equal to or more than a shortest evaluation operation time of the evaluation apparatus and is obtained by adding or subtracting a desired time interval shorter than the shortest evaluation operation time to or from the time interval of each process.

20. A non-transitory computer readable recording medium storing a biological sample evaluation control program for realizing a function of a control apparatus in a biological sample evaluation system comprising a processing apparatus that performs an identical process on a plurality of biological samples of an identical type, an evaluation apparatus that evaluates the plurality of biological samples, and the control apparatus that controls a time interval of each process on each of the biological samples in the processing apparatus and a time interval of each evaluation of each biological sample in the evaluation apparatus, the program causing the control apparatus to execute:
a procedure of setting the time interval of each process to a time interval which is equal to or more than a shortest process operation time of the processing apparatus;
a procedure of setting the time interval of each evaluation to a time interval which is equal to or more than a shortest evaluation operation time of the evaluation apparatus and is obtained by adding or subtracting a desired time interval shorter than the shortest evaluation operation time to or from the time interval of each process;
a procedure of sequentially performing processes on the respective biological samples at the time interval of each process from a reference time; and
a procedure of sequentially evaluating the respective biological samples at the time interval of each evaluation from a time point at which a preset time elapses from the reference time.
